(19) United States
(12) Patent Application Publication
(10) Pub. No.: US 2006/0190089 A1
(43) Pub. Date: Aug. 24, 2006

(54) Internal adaptor for hip acetabular cage

(75) Inventors: Jorge A. Montoya, Summit, NJ (US); Philip Morgan Watt, Cedar Grove, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/062,099

(22) Filed: Feb. 18, 2005

(51) Int. Cl. A61F 2/34

(52) U.S. Cl. 623/22.28; 623/22.25; 623/22.32

(57) ABSTRACT

An acetabular cup assembly for a prosthetic hip joint includes an outer shell for attachment to the acetabulum. The outer shell has an inner surface receiving an adaptor. Complimentary coupling features are provided on the inner surface of the outer shell and the outer surface of the adaptor for coupling the bearing insert to the outer shell. A plurality of adaptors are provided having inclined outer surfaces for engaging an inner surface of the outer shell and having an inner surface for receiving the bearing insert. Each shell has at least one radial extending strap or flange for engaging the bone surrounding the acetabulum. The straps or flanges may be of different sizes and shapes and may be at different locations on the shell. The inner surface of the adaptor includes coupling features be able to receive different design bearing inserts.

### BACKGROUND OF THE INVENTION

This invention relates generally to orthopedics and, in particular, to a versatile reconstruction system associated with an acetabular prosthetic implant.

The replacement of members of a natural hip joint (femoral and acetabular) with prosthetic implants has become widespread and is being accomplished with ever-increasing frequency. The variety of conditions encountered when effecting such implants has led to the use of various bearing materials and attachment methods placed at an optimum position and orientation, as determined by conditions encountered at the site of the implant. The choice of a particular material for the bearing, as well as the size, positioning and orientation of the bearing member, is determined by the surgeon performing the procedure. Usually such choices are made on the basis of a pre-operative assessment of the needs of a particular patient; however, at times the choices are not completed until the implant site actually is being prepared and conditions encountered at the site can be evaluated during the implant procedure itself. Accordingly, it would be advantageous to have available a greater range of interoperative choices, as well as pre-operative choices, so as to enable a surgeon to accommodate the needs of a particular patient as determined by either or both a pre-operative assessment and an evaluation of conditions encountered at a particular implant site, and to do so in a practical manner.

Especially in the case of revision surgical procedures wherein the pelvis has been severely compromised or deteriorated, it is known to use support structures to receive an acetabular prosthetic device. More particularly, this invention relates to outer shells or cages which include wings, straps or flanges to enhance the support of the acetabular prosthesis on the pelvis. The cages include tapered inner surfaces to receive adaptors having tapered outer surfaces. The adaptor has a cavity for receiving a bearing element. Such a system is disclosed in co-pending U.S. application Ser. No. 10/878,787, entitled “INTERNAL FIXATION ELEMENT FOR HIP ACETABULAR SHELL” assigned to the assignee of the present application. The disclosure of that application is incorporated herein by reference.

Another such structure is disclosed in U.S. Pat. No. 5,314,490 to Wagner et al., entitled OUTER CUP FOR AN ARTIFICIAL HIP JOINT SOCKET. According to this patent, an artificial hip joint socket for fastening to a pelvic bone includes a metallic outer cup forming a concavity for receiving a hip, which terminates in an equatorial edge to which supporting flaps are fastened. The flaps include holes to receive bone screws and have preset lines or grooves to enabling preferential bending to provide conformance with the pelvic region surrounding the procedure.

U.S. Pat. No. 5,425,778 relates to an acetabular socket supporting ring having at least one attaching strap for attachment to a corresponding bone portion. This construction has a disadvantage that the support ring must be used in all applications since it receives the outer shell of the acetabular prosthesis.

U.S. Pat. Nos. 5,871,548 and 6,340,370 disclose modular outer shells having wings, straps or flanges which selectively attach to the shell if required.

U.S. Pat. Nos. 6,475,243 and 6,610,097 and European Patent No. 0 773 007 relate to modular acetabular cup systems with inserts which can position bearing liners at various angles to the open face of the outer cup shell. The disclosure of U.S. Pat. No. 6,475,243 is incorporated herein by reference. The prosthetic modular acetabular system of the present invention allows the user to create an outer shell or base (dome and plates or straps) construct for the reconstruction of an acetabulum joint member in revision surgery. This design allows the user to independently set the position of an acetabular liner (polyethylene, metal or ceramic) through the insertion of a metal adaptor or sleeve.

The metal adaptor or sleeve will preferably be available in (0°, 10°, 20°, etc.) inclinations and will connect to the base outer cage construct through a taper junction. The presence of a symmetrical taper, on the outside of the metal sleeve, allows anteversion and placement of the metal sleeve around a circular or 360° range prior to taper locking. A bolt between the sleeve or adaptor and the cage base will ensure locking of the tapers. The main purpose of the design is to obtain independent placement, inclination and fixation of an acetabular bearing liner from the already established base construct by means of the metal sleeve described above.

The present invention allows the base construct and the orientation/inclination of the liner to be independently assessed, set and fixed through a metal adaptor component. The metal adaptor or sleeve component can be easily removed from the base construct and reoriented if necessary according to the user's needs. The procedure for implanting the modular cage is as follows: the shell is placed into the acetabulum and secured with screws through the dome and external plates. This allows a solid foundation to be established. Next, a trial adaptor sleeve is inserted into the shell to establish the correct inclination and orientation of the sleeve. At this point, the bearing liner is also trialed. The correct adaptor or sleeve implant is impacted into the shell to engage the taper junction between the shell and sleeve. A bolt or screw is then inserted into the construct. The bearing liner is trialed for a second time. The proper bearing liner implant is impacted into the cage/sleeve construct.

The present invention provides the surgeon with the ability to choose, either pre-operatively or intra-operatively, an optimum, position and orientation for an acetabular cup assembly to be implanted at a particular implant site, with increased ease and at lowered expense. As such, the present invention attains several objects and advantages, some of which are that it allows the choice of the size, position and orientation of the bearing surface of a bearing member selected for assembly with a particular acetabular shell; increases the range of the bearing size, positioning and orientation, and renders the choices available in a practical manner for either pre-operative or inter-operative selection; allows a surgeon greater latitude in accommodating the needs of different patients while meeting the requirements imposed by various conditions encountered at a particular implant site, and enables appropriate choices to be made intra-operatively, as well as pre-operatively; promotes greater accuracy in the replacement of a natural hip joint, with increased economy; provides a surgeon with the ability to make both pre-operative choices and inter-operative choices from a wider range of options; facilitates the insertion and securement of a selected bearing member within an acetabular shell in appropriate alignment and orientation of the bearing member within the acetabular base, cage, or shell; provides an acetabular cup assembly having accurate sizing, positioning and orientation, with economy of manufacture and use, and long-term reliability.

As used herein, when referring to bones or other parts of the body, the term “proximal” means closer to the heart and the term “distal” means more distant from the heart. The term “inferior” means toward the feet and the term “superior” means towards the head. The term “anterior” means towards the front part of the body or the face and the term “posterior” means towards the back of the body. The term “medial” means toward the midline of the body and the term “lateral” means away from the midline of the body.

### SUMMARY OF THE INVENTION

The above aspects and advantages, as well as further aspects and advantages, are attained by the present invention which may be described briefly as an acetabular cup assembly for receiving a proximal end of a femoral component of a prosthetic hip implant, the femoral component including a head member and a neck member depending from the head member, the acetabular cup assembly having an external cage or shell member with an internal cavity, including a coupling element, an adaptor with an internal cavity and an internal bearing member of a ceramic, metal or polymer for securement within the adaptor cavity to receive the head member of the femoral component for rotational movement within the bearing member. The internal bearing member has an external coupling element and may be selected from a plurality of bearing members which may have different characteristics. The cup assembly may include a metallic outer shell or cage having wings, flanges or straps extending radially outwardly therefrom which cage is provided for reception within the cavity of the acetabulum. The adaptor extends between an upper end and a lower end of the cage and preferably includes a tapered external securing element and an internal coupling element for engaging the external coupling element on the bearing member. The wings, flanges or straps may be modular or integral with the shell or cage. Preferably, the wings, flanges or straps are ductile enough to be bent or deformed by the surgeon to closely conform to adjacent bones. Alternatively, the wings, flanges or straps may have grooves or thinner areas to aid in bending. The outer cage preferably has an internal locking surface for receiving the adaptor. The preferred internal locking surface on the outer shell and the corresponding outer locking surface on the adaptor are preferably mating locking tapered surfaces. In the preferred embodiment, there are multiple adaptors, the tapered surfaces on the adaptors with respect to its open face vary from one adaptor to another with respect to the angle of the tapered surface with the outer cage polar axis. Thus, since the adaptor tapered surface axis is inclined with respect to the cage polar axis, rotation of the adaptor, prior to it being locked within the shell results in multiple orientation of the bearing surface with respect to the cage.

Further, the present invention provides a cage or shell member and adaptor for use in an acetabular cup assembly having an internal polymeric or ceramic bearing member for securement within the adaptor. The internal polymeric or ceramic bearing member is selected from a plurality of bearing members having different characteristics such as bearing surface size for different heads or inclinations. The adaptor member includes an internal wall of its cavity with a coupling element matching on the external wall of the bearing member. A tapered coupling element may be on the adaptor inner surface and would be compatible with the external tapered securing characteristics of the plurality of bearing members such as ceramic bearings. Optionally, a second additional bearing coupling element may be provided within the cavity of the adaptor, the second securing element being compatible with the securing characteristics of other internal bearing members so that a, for example, polymeric bearing element may be introduced directly into the adaptor. The first coupling element and the second optional coupling element being juxtaposed with one another and placed at relative locations such that the effectiveness of each of the first and second securing elements is maintained in the presence of the other of the first and second securing elements, whereby the one and the another of the internal bearing members each is selectable for effective securement within the adaptor member which in turn is selectable for implantation into the outer cage or shell to complete the acetabular cup assembly.

The coupling element or elements formed on the inner surface of the adaptor may also be present on the inner surface of the outer cage. This allows a bearing element to be mounted on the tapered conical inner surface of the outer cage. The outer cage or shell includes outwardly extending wings, flanges or straps for engaging the bone adjacent the acetabulum. The wings or flanges include holes to accommodate bone screws which engage the pelvic bone. The straps or wings may be modular and have mounting features, such as screws which engage corresponding mounting features on the securing elements for their assembly to the hemispherical adaptor body. Such features are well known in the prior art. As discussed above, in addition, the flanges or wings may be bendable to allow the surgeon to conform the shape of the wings or straps to the pelvic bone. This may be done, for example, by making the straps or wings of a deformable material such as a ductile metal or making the material cross-section relatively thin throughout its length or at least at selected areas along the length of the wing or strap.

In addition, the present invention includes a kit of component parts for assembling an acetabular cup assembly having an adaptor secured within an outer shell or cage and an internal bearing member secured within the adaptor member, the kit comprising: a plurality of bearing members having different characteristics such that the acetabular cup assembly selectively is provided with characteristics corresponding to the characteristics of a selected one of the internal bearing members (preferably made of ceramic or polyethylene); a plurality of cage or shell members comprising: an internal cavity having at least one coupling element in the form of a tapered inner surface with a first coupling element within the cavity of the cage member being compatible with the tapered outer surface of a plurality of adaptors; and optionally, a second coupling element within the cavity of the shell member, the second coupling element being compatible with the securing characteristics of at least one of the plurality of internal bearing members; the first and second coupling elements may be juxtaposed with one another and placed at relative locations such that the effectiveness of each of the first and second coupling elements is maintained in the presence of the other of the first and second coupling elements, whereby the one and the another of the internal bearing members each is selectable for effective securement within the outer shell member as the selected one bearing member to complete the acetabular cup assembly. The tapered inner surface of the shell lockingly engages the adaptor which in turn lockingly receives the bearing element. The plurality of adaptors have inner surfaces oriented at different angles or inclinations such as 0°, 10°, 20°, etc. with respect to the polar axis of the outer part spherical cage. Preferably these surfaces are also inwardly tapered i.e., tapered inwardly from the open end towards the polar area of the adaptor to engage complimentary tapered surfaces on the outside of the bearing component.

These and other objects of the invention are set forth in an acetabular cup assembly used in a prosthetic hip joint replacement which has an outer shell for attachment to the acetabulum, a bearing insert portion and an adaptor. The outer shell or cage has a part spherical inner surface and a preferably tapered coupling mechanism adapted to receive the adaptor. The bearing has an external coupling element for engagement with a complementary coupling element on the internal surface of the adaptor. The adaptor has a tapered outer surface for engaging the inner tapered surface of the outer shell or cage with its inner surface adapted for receiving the bearing element. The adaptor tapered surface may be inclined with respect to the tapered shell surface. The shell may have at least one radially extending strap, flange or wing which either conforms with or can be deformed in a manner to conform with the bone surrounding the acetabulum.

The deformable wing, flange or strap is deformable at least in a direction perpendicular to the radial direction and may be oriented at various angles around and extend various lengths with respect to the body of the adaptor. Obviously, the longer the length, the greater the engagement with various areas of the pelvis are possible. The wing, strap or flange may include a reduced cross-section to facilitate deformation or may be made of a ductile material. Alternately, the flange can have a groove or series of grooves to facilitate bending.

The engagement or coupling mechanism between the cage and the outer surface of the adaptor is preferably a complementary conically tapered surface. The surface has a tapered angle so that a locking coupling can be formed between the shell or cage and the adaptor. Preferably, additionally the cage and adaptor includes a threaded coupling which extends in the polar region of either the cage and can threadably couple the cage and adaptor together. Preferably, tightening of the threaded coupling forces locking engagement between the complementary preferably conically tapered surfaces.

The above structure can be supplied in kit form with a variety of shells, adaptors, bearing inserts, which bearings are preferably made of polyethylene and/or ceramic. If ceramic, the bearing may be surrounded by a press-fit metal adaptor. The cages or shells may have one or more wings located in fixed positions around the circumference of the shell or, in fact, may be modular and therefore attachable such as by threaded connectors, such as screws, at any desired angular orientation around the circumference of the shell. Thus, the parts may be selected intraoperatively. This is especially helpful when the pelvis is deformed or otherwise missing natural bone so that shells or cages having straps oriented in various angular locations and of various lengths can be selected from the kit and placed in position usually utilizing screws.

### BRIEF DESCRIPTION OF THE DRAWINGS

FIG. 1 is a bottom view of the assembled acetabular cup system of the present invention;

FIG. 2 is an exploded isometric view of the acetabular cup assembly of FIG. 1 including a polymeric bearing insert, adaptor and outer shell;

FIG. 3 is a cross-sectional view of the exploded system of FIG. 2 along lines 3-3;

FIG. 4 is a cross-sectional view of the assembly of FIG. 1 having an angled adaptor along lines 4-4;

FIG. 5 is a cross-sectional view of the assembly of FIG. 1 with a non-angled adaptor along lines 5-5;

FIG. 6 is a side view of the angled adaptor of FIG. 4;

FIG. 7 is a bottom plan view of the outer acetabular shell of the present invention;

FIG. 8 is an exploded isometric view of the acetabular cup system of the present invention including a ceramic bearing for assembly into shell and adaptor of FIG. 8; and;

FIGS. 9 and 10 show the assembled adaptor and shell of FIG. 8 with an angled and non-angled adaptor with a ceramic bearing insert.

### DETAILED DESCRIPTION

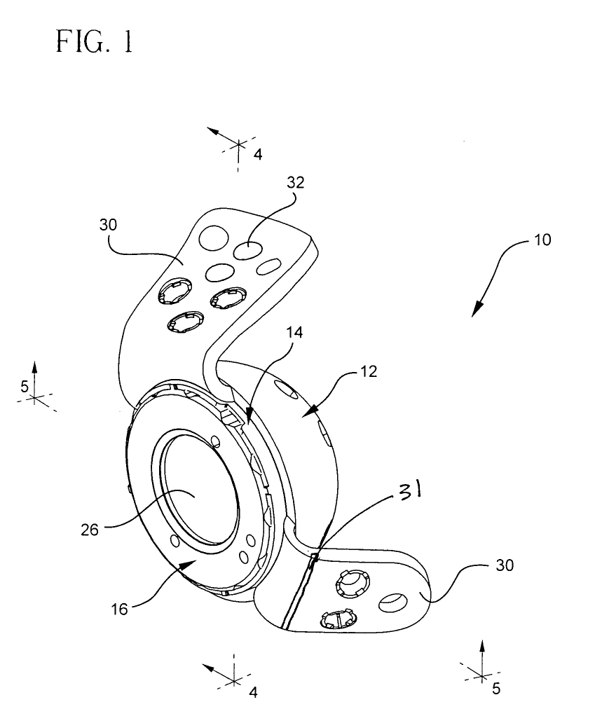

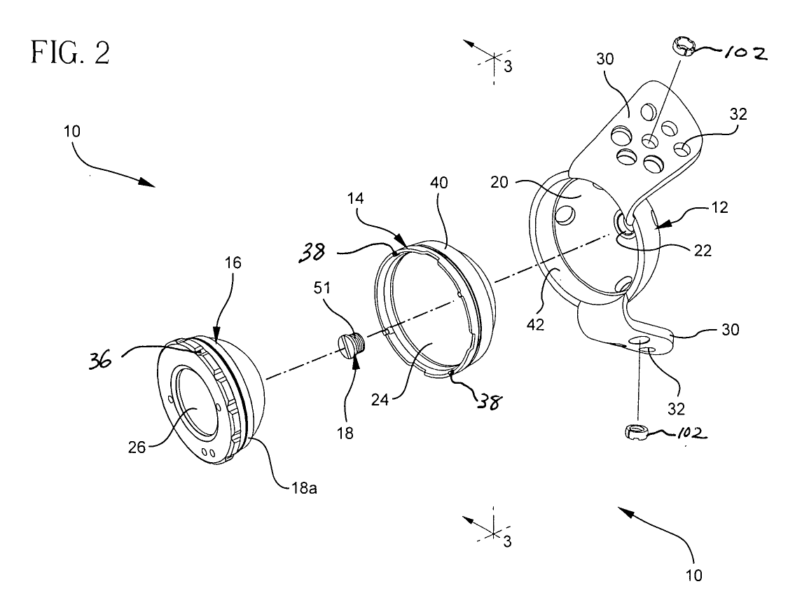

Referring to FIGS. 1 and 2 there is shown an assembled and an exploded isometric view of the preferred acetabular prosthesis of the present invention generally denoted as 10. Assembly 10 consists of an outer shell or cage 12, an adaptor 14, a bearing insert 16 which, as shown in FIG. 2 is made of polyethylene, and a fastening mechanism 18 such as a screw. Adaptor 14 is designed to be inserted within a cavity 20 of shell 12. Connecting element or screw 18 is adapted to be threaded into the polar hole 22 of the shell 20 to thereby clamp the adaptor in position. The polymeric bearing insert 16 is then inserted into the inner cavity 24 of adaptor 14 and locked therein by any convenient known method. The assembled acetabular cup implant 10 of FIG. 2 is shown in FIG. 1. Bearing insert 16 in turn has an internal cavity 26 for receiving the ball shaped head of a femoral component (not shown). Preferably the adaptor outer surface and shell inner surface have complimentary locking tapers. Tightening of screw 18 ensures the solid locking of the two parts together by the tight engagement of the tapers.

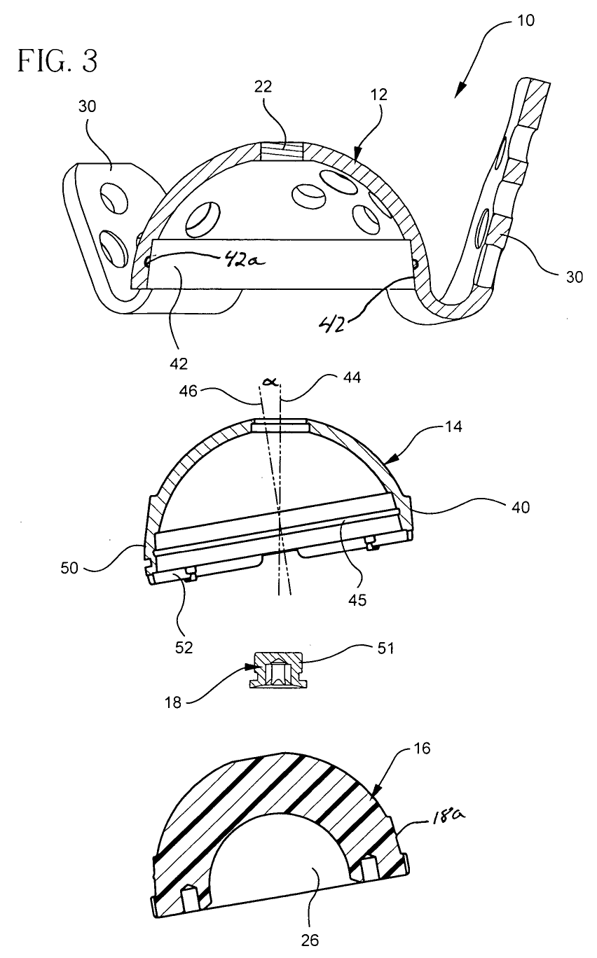

Also shown on shell or cage 12 in FIGS. 1 and 2 are the metal straps, flanges, or wings 30 which may be deformed to conform to the pelvis surrounding the acetabulum. Straps 30 are preferably deformable and include a series of holes 32 to accommodate bone screws. Straps 30 may have a reduced cross-section preferable in the form of a groove 31 to assist bending the strap. FIG. 3 is a cross-sectional view of the exploded view of FIG. 1 showing an angled adaptor 14. the angled adaptor will be discussed in further detail below.

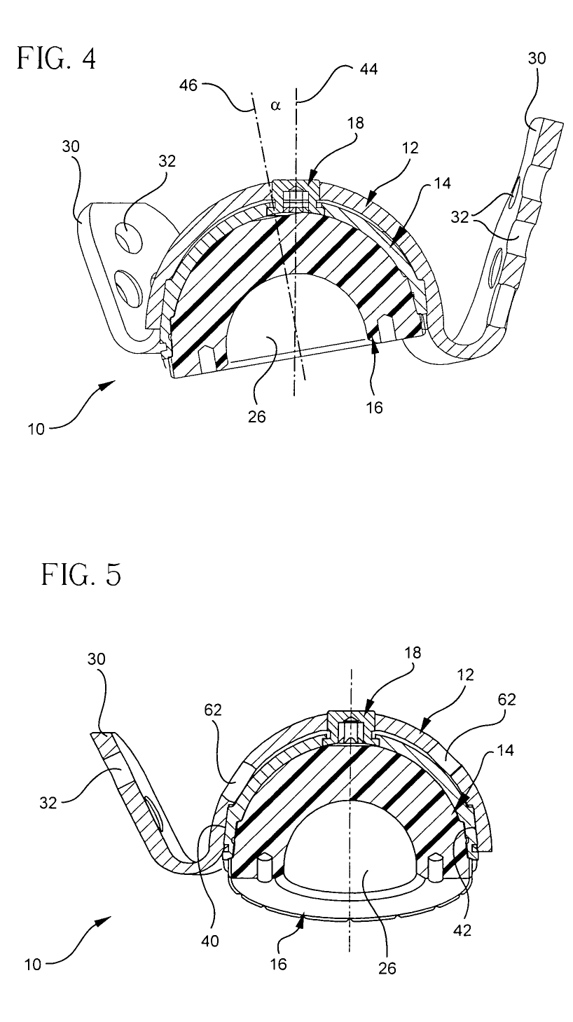

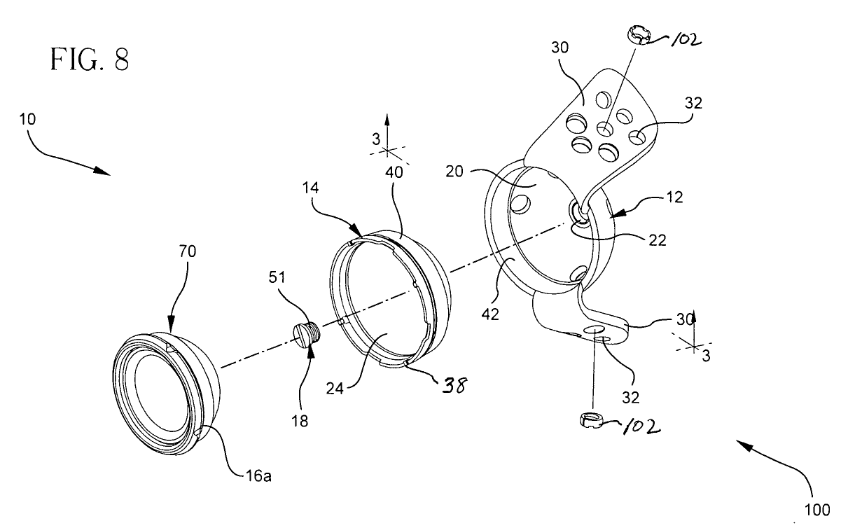

Referring to FIGS. 4 and 5 there is shown cross-sectional views of the assembly of FIG. 1 along lines 3-3 and 4-4 respectively. In the embodiments of FIGS. 4 and 5 a polyethylene bearing insert 16 is shown mounted within adaptor 14. In the preferred embodiment, a polyethylene bearing insert may be locked into the adaptor 14 in any well known manner including having a deformable ring or lip 18a on an external surface thereof and/or by using interlocking tapered surfaces. In addition, anti-rotation elements such as cutouts 36 (FIG. 2) may engage on inwardly extending detent 38 which, when engaged, prevent the relative rotation between adaptor 14 and bearing 16. If a ceramic bearing 16a (shown in FIG. 8-10) is utilized, the outer surface of the ceramic bearing is normally tapered to engage the taper on the inner wall of adaptor 14 to form a locking connection in the manner of a Morse taper as discussed above with respecting to shell 12 and adaptor 14. This tapered outer surface may be on a metal ring press-fit onto the outer ceramic bearing surface during manufacture.

As best seen in FIGS. 4, 5, 9 and 10 adaptor 14 is mounted within outer shell 12 via mating tapered surfaces 40 and 42 on the adaptor and outer shell respectively. As can be seen in FIG. 4 the polar axis through the open face of adaptor 14 represented by axis 44 is inclined at an angle ∂ of, for example 10°, 20°, and 30° from the polar axis 46 of the open face of outer shell or cage 12. As shown in FIGS. 5 and 10 an adaptor which orients the bearing at 0° (co-axial) with the cage or shell polar axis may also be provided. Since a plurality of adaptors 14 are provided at varying angular offsets (in 5 or preferably 10° increments) between axis 44 and 46, the face of bearing 16 can be oriented by the surgeon in a manner intraoperatively which best prevents dislocation of the hip joint. In addition, because of the use of conical Morse type tapers 40, 42 the adaptor, and therefore bearing insert 16, 16a can be rotated about axis 46 to vary the orientation of the cavity 26 within bearing member 16. When a polyethylene insert 16 is used adaptor 14 includes a groove 45 to receive a typical lip or protrusion around the outer circumference of a polyethylene bearing. As shown in FIG. 5a adaptor 14 includes an inner tapered surface 47 which extends on both sides of groove 45. In addition, the adaptor has a hole 49 to accommodate a head 51 of screw 18. Preferably hole 49 is unthreaded. When assembled screw 18 is inserted through hole 49 of adaptor 14 into threaded bore 22 and tightened. Head 51 engages the inner surface of adaptor 14 around hole 49. Complete tightening ensures the tight engagement of tapered locking surfaces 40 and 42.

Referring to FIG. 5 there is shown the adaptor 14 of FIGS. 3 and 4 disassembled from the cup assembly 10. Adaptor 14 is shown to have a hooded area 50 which is oriented around a face 52 of adaptor 14 at an angle with respect to the equator 54 of tapered surface 40. The angle between equator 54 and face 52 is represented by the angle alpha in FIG. 5. The purpose of the hooded area 50 is to prevent dislocation of the hip by providing more coverage to the head of the femoral stem in a preferred anatomical orientation. This results in the axis 46 of the open end of adaptor 14 being inclined at the angle ∂ with polar axis 44 of outer shell 12.

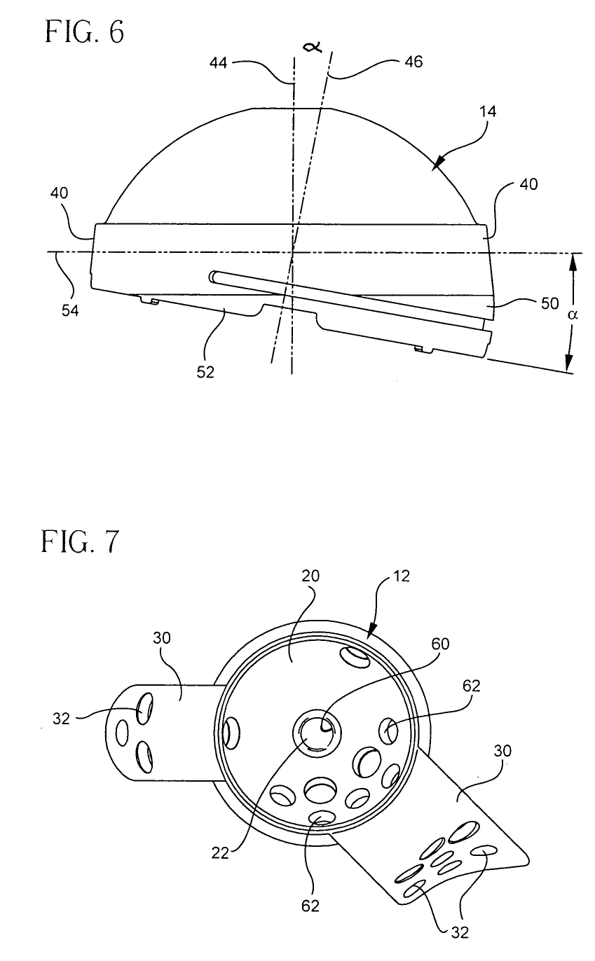

Referring to FIGS. 6 and 7 there is shown the adaptor 14 and the outer shell 12 prior to assembly within the cup system 10. In the preferred embodiment, outer shell 12 includes a polar hole 22 with threads 60 to receive coupling screw 18 and a plurality of typical holes 62 adapted to receive bone screws (not shown) which are used to mount outer shell 12 within the natural acetabulum. Fewer than all Both FIGS. 4 and 7 show the flanges, straps, or wings which are used as additional attachment element for attaching shell or cage 12 to the bone surrounding the acetabulum. Preferably wings or straps 30 are flexible or ductile enough to be deformed by the surgeon. As best seen in FIG. 3 the inner surface of the preferred outer shell or cage 12 includes the tapered surface 42 which extends around the circumference of the inner cavity 20 thereof. As discussed above, tapered surface 42 can include an auxiliary attachment means such as a groove 42a so that a bearing insert may be coupled directly to the outer cage 12 without the use of adaptor 14.

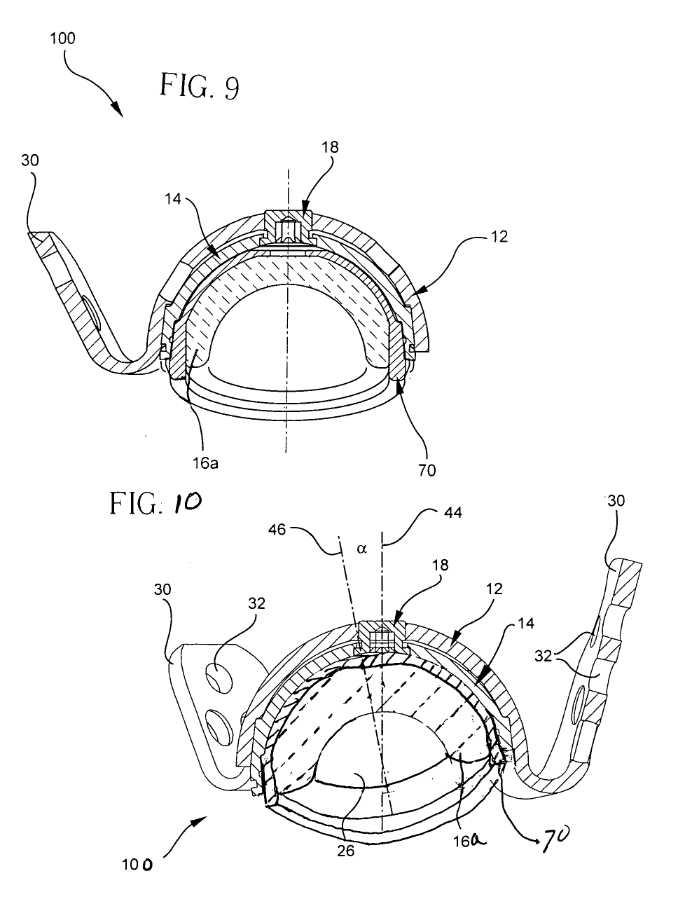

Referring to FIGS. 8, 9 and 10, there is shown, in FIG. 8, an exploded view of outer shell 12, and a ceramic bearing 16a. FIGS. 9 and 10 show an assembled view of FIG. 8 with both an angled (FIG. 9) and non-angled adaptor (FIG. 10) Tapered surfaces 40 and 42 locked together via screw 18 threaded into the threads of hole 60 of outer cage 12. FIGS. 9 and 10 show ceramic bearing assembly 16a (made up of the ceramic bearing and shrink fit metal sleeve) which may be used within the same adaptor 14 as used with polyethylene bearing 16 and coupled via a tapered locking system. Ceramic bearing assembly 16a includes recesses 70 for engaging detent 38 on adaptor 14 to prevent the relative rotation therebetween. The insert of FIGS. 3, 4, and 5 is preferably an ultra-high molecular weight polyethylene insert which is identical in all respects to various well known inserts and may be coupled to adaptor 14 either via tapered surface or by a protrusion or lip 18a extending around the circumference of the bearing insert which engages groove 45 on insert 14.

The preferred method of use of the acetabular cup system 10 is for the surgeon to initially implant outer cage 12 in a desired location utilizing a plurality of bone screws. Bone screws may be used in both the holes 62 in the part-spherical shell 12 and also in holes 32 of straps 30. The screw may be inserted in a mono-axial fashion into holes 32, 62 or holes 32, 62 may include part-spherical walls having a part-spherical ring 102 allowing for polyaxial orientation of the screw. Such a design is shown in U.S. Patent application filed this date listing Gregory Plaskon as inventor and assigned to the assignee of the present invention. The outer shell 12 may be chosen from a series of different size shells depending on the size of the acetabulum or location of the straps 30. Once implanted, an adaptor 14 can be chosen from a plurality of adaptors and mated with outer shell 12 via the interlocking of tapered surfaces 40 and 42. The plurality of adaptors 14 have polar axis 46 located at different angles to the polar axis of 44 of shell 12 as discussed above and, on insertion, can be oriented in a manner which best corresponds to the anatomy of the patient. Once properly oriented the surgeon inserts screw 18 through hole 49 and into hole 22 and locks the shell/adaptor assembly together. Alternatively the adaptor and bearing may be impacted into place.

The surgeon may then insert the desired ceramic or polyethylene bearing liner 16, 16a. Alternatively, if screw 18 is not used, the bearing liner 16 can be assembled into the adaptor 14 prior to insertion of the adaptor 14 into outer cage 12.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An acetabular cup assembly for a prosthetic hip joint comprising:
a part-spherical outer shell for attachment to the acetabulum said shell having an inner surface having a tapered locking surface and an outer surface having at least one radially extending strap, the outer shell having an open end defining a central polar axis;
a bearing insert portion;
an adaptor having a tapered outer surface for engaging the inner surface of the outer shell and an inner surface for receiving the bearing insert portion, said adaptor having an open end defining a polar axis inclined with respect to the polar axis of the outer shell.

2. The acetabular cup assembly as set forth in claim 1 wherein said strap is deformable at least in a direction perpendicular to said radial direction.

3. The acetabular cup assembly as set forth in claim 2 wherein said at least one strap includes a reduced cross-section to facilitate said deformation.

4. The acetabular cup assembly as set forth in claim 3 wherein said reduced cross-section is formed by a groove.

5. The acetabular cup assembly as set forth in claim 1 wherein said inner surface of said shell and said outer surface of said adaptor have complimentary Morse tapered locking surfaces.

6. The acetabular cup assembly as set forth in claim 5 wherein said adaptor and said shell included a threaded connection for lockingly engaging said complimentary tapered surfaces.

7. The acetabular cup as set forth in claim 5 wherein said inner surface of said adaptor has a coupling structure for engaging a complementary locking structure on an outer surface of said bearing element.

8. The acetabular cup assembly as set forth in claim 1 wherein said straps are integral with said outer shell.

9. The acetabular cup assembly as set forth in claim 1 wherein said polar axis of said adapter is inclined from 10° to 30° from the polar axis of said shell.

10. A kit for resurfacing an acetabulum comprising:
at least one part-spherical shell having an outer surface for contacting the acetabulum and an inner surface with an open end defining a polar axis, said shell including at least one radially outwardly extending strap;
a plurality of adaptors having an outer surface for engaging the inner surface of said shell; the adaptor having an open end defining a polar axis, said polar axis of at least one adaptor being inclined to the polar axis of the shell upon engagement of said inner and outer surfaces, said flanged insert having an inner surface with an engagement feature;
at least one bearing insert for insertion into said adaptor and having a locking element for engaging the engagement feature of said adaptor.

11. The kit as set forth in claim 10 wherein said engaging inner surface of said shell and said outer surface of said adaptor having complimentary tapered locking surfaces.

12. The kit as set forth in claim 11 wherein said adaptor and said shell included a threaded connection for lockingly engaging said complimentary tapered surfaces.

13. The kit as set forth in claim 12 wherein said adaptors each include an internal surface for coupling to said at least one bearing member.

14. The kit as set forth in claim 13 wherein the bearing member includes a rib projecting from the bearing member and the internal coupling elements of said adaptor has a recess for receiving the rib of the bearing member.

15. The kit as set forth in claim 14 wherein the recess in the adaptor is intermediate ends of said tapered internal surface on said adaptor.

16. The kit as set forth in claim 10 wherein the adaptor member includes a tapered external securing surface, and the shell includes a tapered internal securing surface, the external securing surface and the internal securing surface being complementary tapered configurations for interlocking in response to seating engagement of the complementary tapered configurations.

17. The kit as set forth in claim 10 wherein said straps are selectably attachable to said outer shell adjacent said open end.

18. The kit as set forth in claim 10 wherein said kit comprises at least three adaptors each having a polar axis inclined at a different angle to said polar axis of said shell.

19. The kit as set forth in claim 18 wherein said adaptor angles are 10°, 20° and 30° respectively.

20. The acetabular cup assembly as set forth in claim 1 wherein at least one of the shell or straps has apertures for bone screws including a rotatable ring for receiving a bone screw allowing polyaxial rotation of the screw.
